(19) United States
(12) Patent Application Publication
(10) Pub. No.: US 2007/0167714 A1
(43) Pub. Date: Jul. 19, 2007

(54) System and Method For Bronchoscopic Navigational Assistance

(75) Inventors: Atilla Peter Kiraly, Plainsboro, NJ (US); Carol L. Novak, Newtown, PA (US)

Correspondence Address: SIEMENS CORPORATION;INTELLECTUAL PROPERTY DEPARTMENT, 170 WOOD AVENUE SOUTH, ISELIN, NJ, 08830, US

(73) Assignee: SIEMENS CORPORATE RESEARCH, INC., Princeton, NJ (US)

(21) Appl. No.: 11/566,746

(22) Filed: Dec. 5, 2006

Related U.S. Application Data
(60) Provisional application No. 60/742,995, filed on Dec. 7, 2005.

(51) Int. Cl. A61B 5/05

(52) U.S. Cl. 600/407; 600/425

(57) ABSTRACT

A computer-based method for bronchoscopic navigational assistance, including: receiving first image data of a patient's lungs, the first image data acquired before a bronchoscopy is performed; receiving second image data of a portion of one of the patient's lungs that includes a bronchoscope, the second image data acquired during the bronchoscopy; and performing image registration between the first image data and the second image data to determine a global location and orientation of the bronchoscope within the patient's lung during the bronchoscopy.

### CROSS-REFERENCE TO RELATED APPLICATION

This application claims the benefit of U.S. Provisional Application No. 60/742,995, filed Dec. 7, 2005, a copy of which is herein incorporated by reference.

### BACKGROUND OF THE INVENTION

1. Technical Field

The present invention relates to bronchoscopic navigation, and more particularly, to a system and method for bronchoscopic navigational assistance.

2. Discussion of the Related Art

Bronchoscopic navigation planning generally involves the manual review of slices of two-dimensional (2D) data from high-resolution computed tomography (HRCT) scanners. Traditionally, a navigation path to any lung abnormality was determined solely from this series of 2D slices. This process, however, has proven to be time consuming and can often lead to inaccurate biopsies for less experienced bronchoscopic operators.

Recently, virtual bronchoscopy (VB) has enabled three-dimensional (3D) visualization of the airways for improved path planning. Basic VB allows one to virtually navigate through the airways in advance of the actual bronchoscopy. VB can provide a map of necessary airway paths to be traversed during the bronchoscopy to reach locations of target points. The location of the target points or pathologies can also be incorporated into the rendering. Although it is possible to view a particular path in a cine loop, this approach is of limited aid to the bronchoscopist during the bronchoscopy, and thus, only serves as a guideline.

Another approach for improved path planning is to acquire a physical model of the bronchoscope. This model is then combined with the model of a patient's airways to determine the position and orientation of the bronchoscope at the location of a pathological site. The physical insertion procedure can then be derived and provided as a guideline for the insertion procedure to be used by a bronchoscopist during the bronchoscopy. Although capable of providing a step-by-step Guideline for the bronchoscopy, this method is incapable of providing a real-time location of the bronchoscope within the patient.

Three methods currently offer guidance during a bronchoscopy. These methods allow a bronchoscopist to see their current location within a scanned CT volume.

The first method requires that the bronchoscopy be performed within the CT scanning room. Here, a CT scan is taken during the bronchoscopy to see the location of the scope within a patient's airways. A disadvantage of this method is that it must be performed in the CT scanning room and that it requires a temporary halt of the bronchoscopy to obtain the CT scan. In addition, the newly acquired CT data must then be manually analyzed to further plan the navigation. Further, acquiring the CT scan can expose the bronchoscopic staff to radiation. This procedure can also be expensive as it ties up the CT scanner during the entire bronchoscopy.

The second method involves using a positional sensor that gives real-time updates regarding the location of the tip of the bronchoscope. However, the use of positional sensors requires modification to the bronchoscope and the careful placement of calibration markers on and around a patient. These sensors tend to drift in positional reading, thus creating an accumulation of errors during the bronchoscopy. In addition, the initial calibration can be difficult to perform.

The third method involves capturing a bronchoscopic video and matching it to virtual views obtained from a VB system based on a planning CT scan to estimate the location of the bronchoscope within a patient's airways. In this method, an optical model of the bronchoscope is determined and used to remove the effect of the bronchoscope's lens on the video data. The processed video data is then compared to renderings of the planning data. The comparisons determine a score of how close the two images are to each other. Here, the goal is to determine the location and orientation of the bronchoscope by finding the most similar virtual view with the planning data to the actual video data. Hence, a total of six degrees of freedom must be determined.

Although video-based methods offer the least intrusive method for assisted navigation, these methods do not always achieve real-time performance since multiple locations and orientations must be searched, thus making it potentially necessary for the bronchoscopist to wait for the location to be determined. In addition, fast movement of the bronchoscope and “bubble frames”, which are frames of the video containing shiny air-filled bubbles, can create difficulties when tracking. Further, locations without distinctive features, such as those within a bronchus not near a bifurcation or wall, can also create situations where these methods cannot provide a correct match.

Recently, a combined approach of video-based tracking and physical sensor tracking has been proposed. This combined approach has led to real-time capabilities in tracking. Here, a positional sensor is used to speed up video tracking to a real-time level by constraining the search range for the location and orientation of the bronchoscope. However, the drifting of sensors on a patient can cause errors in the calculations, and thus, modifications must he made to the bronchoscope. In addition, precisely locating and calibrating the sensors in relation to the patient and CT data can be difficult.

### SUMMARY OF THE INVENTION

In an exemplary embodiment of the present invention, a computer-based method for bronchoscopic navigational assistance, comprises: receiving first image data of a patient's lungs, the first image data acquired before a bronchoscopy is performed; receiving second image data of a portion of one of the patient's lungs that includes a bronchoscope, the second image data acquired during the bronchoscopy; and performing image registration between the first image data and the second image data to determine a global location and orientation of the bronchoscope within the patient's lung during the bronchoscopy.

The first image data and the second image data are acquired by using a three-dimensional (3D) imaging technique. The first image data is a computed tomography (CT) volume. The second image data includes one or more slices of a CT volume. The second image data includes a tip of the bronchoscope.

The method further comprises identifying the bronchoscope by segmenting the bronchoscope in the second image data during the bronchoscopy. The method further comprises identifying an airway tree and a location of a potential or actual pathology from the first image data before the bronchoscopy is performed. The method further comprises superimposing the airway tree and the location of a potential or actual pathology from the first image data onto the second image data during the bronchoscopy. The method further comprises performing a virtual bronchoscopy on the second image data after superimposing the airway tree and the location of a potential or actual pathology from the first image data onto the second image data. The method further comprises: subtracting the bronchoscope from the second image data by segmenting the bronchoscope in the second image data during the bronchoscopy; and performing a virtual bronchoscopy on the second image data after superimposing the location of a potential or actual pathology from the first image data onto the second image data. The method further comprises fusing the first image data with the second image data.

In an exemplary embodiment of the present invention, a method for real-time bronchoscopic navigational assistance, comprises: receiving image data of a patient's lungs, the image data acquired before a bronchoscopy is performed; tracking a current global location and orientation of a bronchoscope in one of the patient's lungs by using an optical model and a physical model of the bronchoscope and real-time video of the bronchoscope during the bronchoscopy; and automatically updating the global location and orientation of the bronchoscope in relation to the image data during the bronchoscopy.

The image data is acquired by using a 3D imaging technique.

The method further comprises identifying an airway tree and a location of a potential or actual pathology from the image data before the bronchoscopy is performed. The method further comprises constraining a search space for a subsequent global location and orientation of the bronchoscope by using the current global location and orientation of the bronchoscope during the bronchoscopy. The method further comprises constraining a search space for a subsequent global location and orientation of the bronchoscope by using a pre-selected path to the location of a potential or actual pathology during the bronchoscopy. The method further comprises constraining a search space for a subsequent global location and orientation of the bronchoscope by using a segmentation of the airway tree during the bronchoscopy. The method further comprises constraining a search space for a subsequent global location and orientation of the bronchoscope by using a depth sensor.

In an exemplary embodiment of the present invention, a system for bronchoscopic navigational assistance, comprises: a memory device for storing a program; a processor in communication with the memory device, the processor operative with the program to: receive first image data of a patient's lungs, the first image data acquired before a bronchoscopy is performed; receive second image data of a portion of one of the patient's lungs that includes a bronchoscope, the second image data acquired during the bronchoscopy; and perform image registration between the first image data and the second image data to determine a global location and orientation of the bronchoscope within the patient's lung during the bronchoscopy.

The first image data and the second image data are received from a 3D imaging device. The processor is further operative with the program to display the global location and orientation of the bronchoscope within the patient's lung. The global location and orientation of the bronchoscope within the patient's lung is displayed on a computer or television monitor.

In an exemplary embodiment of the present invention, a system for real-time bronchoscopic navigational assistance, comprises: a memory device for storing a program; a processor in communication with the memory device, the processor operative with the program to: receive image data of a patient's lungs, the image data acquired before a bronchoscopy is performed; track a current global location and orientation of a bronchoscope in one of the patient's lungs by using an optical model and a physical model of the bronchoscope and real-time video of the bronchoscope during the bronchoscopy; and automatically update the global location and orientation of the bronchoscope in relation to the image data during the bronchoscopy.

The image data is received from a 3D imaging device. The processor is further operative with the program to display the automatically updated global location and orientation of the bronchoscope within the patient's lung. The global location and orientation of the bronchoscope within the patient's lung is displayed on a computer or television monitor.

In an exemplary embodiment of the present invention, a computer-based method for endoscopic navigational assistance, comprises: receiving first image data of region of interest inside a patient, the first image data acquired before an endoscopy is performed; receiving second image data of a portion of the region of interest that includes an endoscope, the second image data acquired during the endoscopy; and performing image registration between the first image data and the second image data to determine a global location and orientation of the endoscope within the region of interest during the endoscopy.

In an exemplary embodiment of the present invention, a method for real-time endoscopic navigational assistance, comprises: receiving image data of a region of interest inside a patient, the image data acquired before an endoscopy is performed; tracking a current global location and orientation of an endoscope in a portion of the region of interest by using an optical model and a physical model of the endoscope and real-time video of the endoscope during the endoscopy; and automatically updating the global location and orientation of the endoscope in relation to the image data during the endoscopy.

The foregoing features are of representative embodiments and are presented to assist in understanding the invention. It should be understood that they are not intended to be considered limitations on the invention as defined by the claims, or limitations on equivalents to the claims. Therefore, this summary of features should not be considered dispositive in determining equivalents. Additional features of the invention will become apparent in the following description, from the drawings and from the claims.

### BRIEF DESCRIPTION OF THE DRAWINGS

FIG. 1 illustrates a method for bronchoscopic navigational assistance according to an exemplary embodiment of the present invention;

FIG. 2 illustrates a method for real-time bronchoscopic navigational assistance according to an exemplary embodiment of the present invention; and

FIG. 3 illustrates a system for bronchoscopic/real-time bronchoscopic navigational assistance according to an exemplary embodiment of the present invention.

### DETAILED DESCRIPTION OF EXEMPLARY EMBODIMENTS

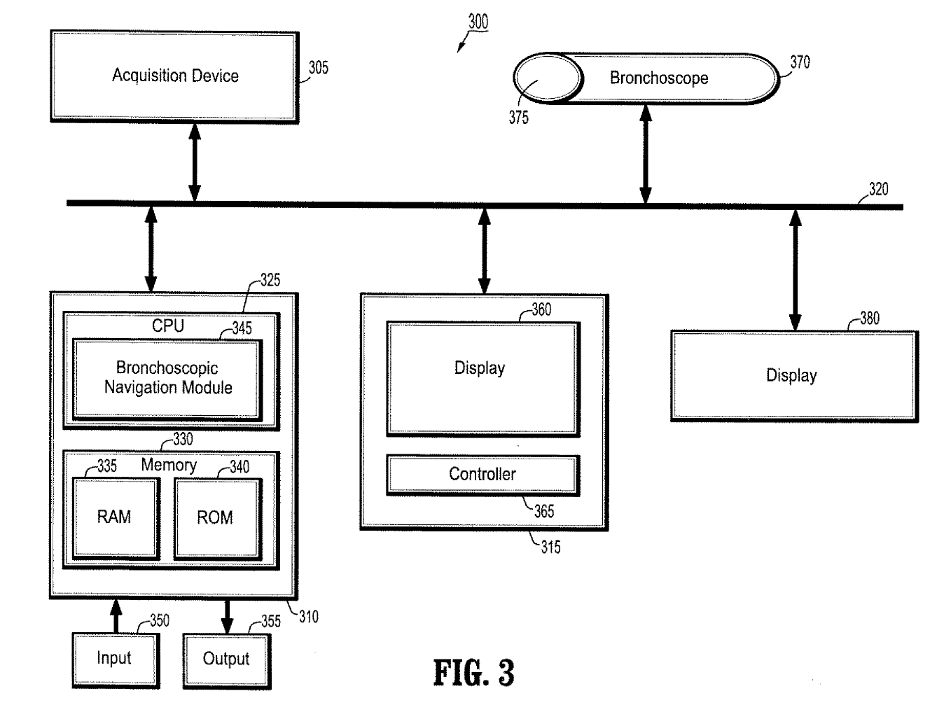

FIG. 3 is a block diagram illustrating a system 300 for bronchoscopic/real-time bronchoscopic navigational assistance according to an exemplary embodiment of the present invention. As shown in FIG. 3, the system 300 includes an acquisition device 305, a PC 310, an operator's console 315, a bronchoscope 370 and a display 380 connected over a wired or wireless network 320.

The acquisition device 305 may be a computed tomography (CT) imaging device or any other three-dimensional (3D) high-resolution imaging device such as a magnetic resonance (MR) scanner.

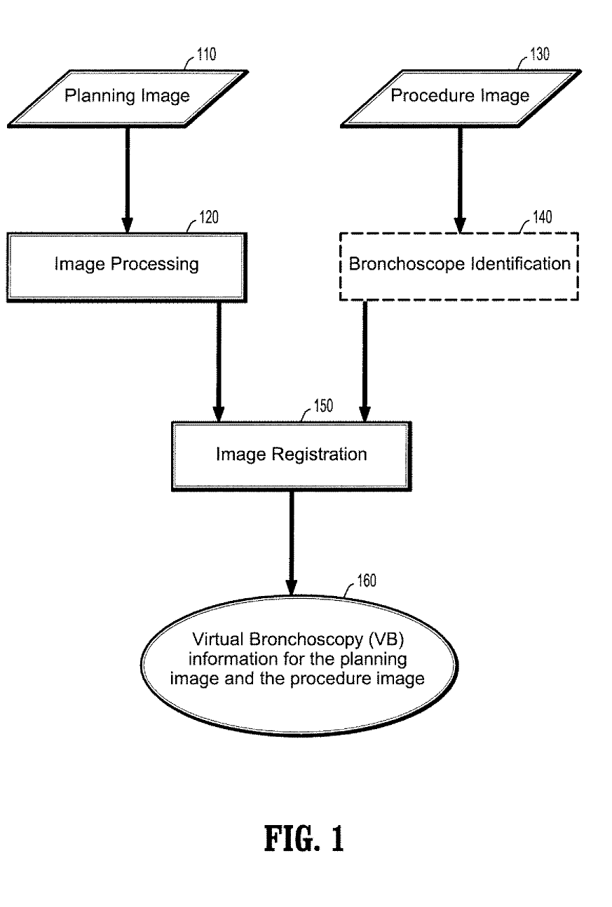

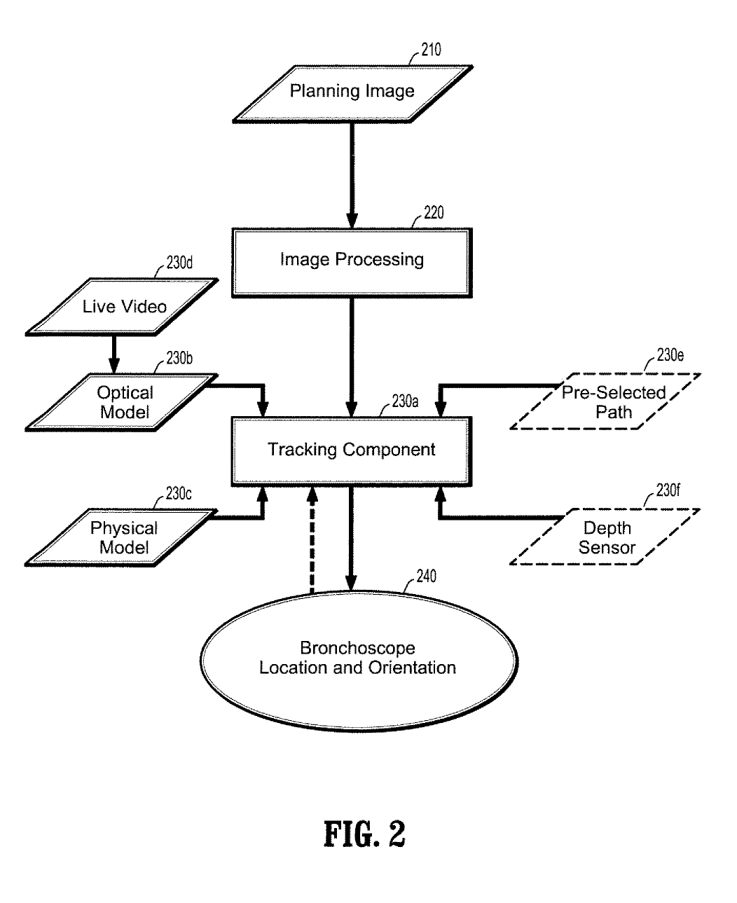

The PC 310, which nay be a portable or laptop computer, includes a CPU 325 and a memory 330 connected to an input device 350 and an output device 355. The CPU 325 includes a bronchoscopic navigation module 345 that includes one or more methods for bronchoscopic/real-time bronchoscopic navigation to be discussed hereinafter with reference to FIGS. 1 and 2. Although shown inside the CPU 325, the bronchoscopic navigation module 345 can be located outside the CPU 325.

The memory 330 includes a RAM 335 and a ROM 340. The memory 330 can also include a database, disk drive, tape drive, etc., or a combination thereof. The RAM 335 functions as a data memory that stores data used during execution of a program in the CPU 325 and is used as a work area. The ROM 34 functions as a program memory for storing a program executed in the CPU 325. The input 350 is constituted by a keyboard, mouse, etc., and the output 355 is constituted by an LCD, CRT display, printer, etc.

The operation of the system 300 can be controlled from the operator's console 315, which includes a controller 365, e.g., a keyboard, and a display 360. The operator's console 315 communicates with the PC 310 and the acquisition device 305 so that image data collected by the acquisition device 305 can be rendered by the PC 310 and viewed on the display 360. The PC 310 can be configured to operate and display information provided by the acquisition device 305 absent the operator's console 315, by using, e.g., the input 350 and output 355 devices to execute certain tasks performed by the controller 365 and display 360.

The operator's console 315 may further include any suitable image rendering system/tool/application that can process digital image data of an acquired image dataset (or portion thereof) to generate and display images on the display 360. More specifically, the image rendering system may be an application that provides rendering and visualization of medical image data, and which executes on a general purpose or specific computer workstation. The PC 310 can also include the above-mentioned image rendering system/tool/application.

The bronchoscope 370 is a slender tubular instrument with a small light 375 on the end for inspection of the interior of the bronchi of a patient. Images of the interior of the bronchi are transmitted by small clear fibers in the bronchoscope 370 for viewing on the display 380.

FIG. 1 illustrates a method for bronchoscopic navigational assistance according to an exemplary embodiment of the present invention. As shown in FIG. 1, a planning image is acquired from a patient (110). This is done, for example, by scanning the patient's chest using the acquisition device 305, in this example a computed tomography (CT) scanner, which is operated at the operator's console 315, to generate a series of 2D image slices associated with the patient's chest. The 2D image slices are then combined to form a 3D image of the patient's lungs, which are stored in the memory 330 and/or viewed on the display 360.

Once the planning image is acquired, an airway tree in the lungs and/or locations of interest such as potential or actual pathologies are identified (120). The airway tree and locations of interest are identified, for example, by performing a segmentation thereof. The segmentation can be performed manually or automatically through several different methods. In one exemplary method, the segmentation can be automatically performed as described in Kiraly A. P., McLennan G., Hoffman E. A., Reinhardt J. M., and Higgins W. E., Three-dimensional human airway segamentation methods for clinical virtual bronchoscopy. Academic Radiology, 2002. 9(10): p. 1153-1168. A copy of this reference is incorporated by reference herein in its entirety.

It is to be understood that prior to or after the segmentation of the airway tree and/or locations of interest, the locations can be manually marked in the planning image. The locations of interest can be manually marked, for example, by identifying a suspicious location in the image and marking it with a cursor or stylus pen or by selecting an area including the suspicious location by using a mouse or other suitable selection means.

Given the planning image and the marked or segmented locations of interest, a bronchoscopy is then performed on the patient. In this embodiment, a procedure image is acquired from the patient (130). This done, for example, by using the same techniques described above for step 110; however, here, the bronchoscope 370 has already been inserted into the patient's bronchi by a bronchoscopist. Thus, the procedure image includes the bronchoscope 370.

At this time, the bronchoscope 370 can be identified via segmentation from the procedure image (140). This is done, for example, by performing a region growing on a region of high density within the airways. This segmentation can be used to determine the location and orientation of the bronchoscope 370 within the procedure image. It is to be understood that this step is optional.

Next, image registration is performed between the planning image and the procedure image (150). This is done by performing any of a variety of image registration techniques. For example, several key points can be selected between the two images and from these points a deformable mapping can be computed.

With the image registration complete, a global location and orientation of the bronchoscope 370 within the patient's lung during the bronchoscopy is determined (160). For example, given the location of the bronchoscope 370 within the procedure image, the deformable mapping computed above can then be used to find the location of the bronchoscope 370 in the planning image. In order to infer the orientation of the bronchoscope 370 in the planning image, an orientation of the bronchoscope 370 must be determined from the procedure image.

Depending on what is required, several options exist at this stage. In one option, for example, the marked or segmented locations of interest in the planning image can be superimposed onto the procedure image. In the alternative, the procedure image can be superimposed onto the marked or segmented locations of interest. In either case, the bronchoscopist can more precisely know where to move the bronchoscope 370 to perform, for example, a biopsy. In addition, the bronchoscopist or a radiologist can more quickly reinterpret the resulting image given the marked locations of interest.

In another option, a virtual bronchoscopy (VB) can be performed on the procedure image that includes the locations of interest superimposed thereon to illustrate to the bronchoscopist the orientation of the bronchoscope 370 and the locations of interest. Here, the remainder of a path, for example, to one of the locations of interest, can be presented in a cine loop.

In yet another option, a VB can again be performed on the procedure image; however, here, the bronchoscope 370 can be subtracted from the image through segmentation. If the procedure image lacks enough resolution and field of view to allow for adequate rendering, the planning image can be fused with the procedure image using registration for a better rendering.

In accordance with this embodiment, a CT scan is used during the bronchoscopy along with VB and image registration. Although this embodiment requires that the bronchoscopy be performed in a CT room, the image processing and registration allow for accurate determination of the location of a pathology in relation to a bronchoscope. Further, this embodiment requires no changes to the bronchoscopy and only requires that a processing computer of VB system obtain a copy of the procedure image.

FIG. 2 illustrates a method for real-time bronchoscopic navigational assistance according to an exemplary embodiment of the present invention. As shown in FIG. 2, a planning image is acquired from a patient (210). This is done, for example, by using the same techniques described above for step 110. Once the planning image is acquired, an airway tree in the lungs and/or locations of interest such as potential or actual pathologies are identified (220). This is done, for example, by using the same techniques described above for step 120.

Given the planning image and the marked or segmented locations of interest, a bronchoscopy is then performed on the patient. In this embodiment, a tracking component is used to track a current global location and orientation of the bronchoscope 370 inside the patient (230a). This is done, for example, by using an optical model (230b) of the bronchoscope 370, a physical model (230c) of the bronchoscope 370 (e.g., the actual bending and size properties of the bronchoscope 370) and live video (230d) of the bronchoscope 370.

As previously discussed with regard to existing video-based methods, the goal is to solve for six degrees of freedom, in other words, the position and orientation of the bronchoscope 370. In these methodologies, only an optical model of a bronchoscope is used to better match a virtual rendered view. However, in this embodiment, the physical model (230c) of the bronchoscope 370 is also used to constrain possible locations and orientations of the bronchoscope 370. These further constraints added by the physical model (230c) limit the region of possibilities for the location and orientation of the bronchoscope 370. Once the tracking component has analyzed this data, the global location and orientation of the bronchoscope 370 in relation to the planning image are automatically updated and then displayed, for example, on the display 380 (240).

It is to be understood that given the physical (230c) and optical models (230b) of the bronchoscope 370, once an initial position of the bronchoscope 370 is established, these model parameters can be constrained for future matches. Thus, by using the additional constraints of the physical model (230c), the previous parameters of the physical model (230c) and the previous orientation, the search space for a matching frame can be significantly reduced. The search space is, for example, the locations and orientations where a specific X,Y,Z location is found within the planning image along with a specific orientation. Since the video (230d) is compared to virtual rendered views from the dataset to determine the optimal location and orientation of the bronchoscope 370, without a constrained search space, one would have to look at every location within the planning image and every orientation to find the most likely match.

In an alternative embodiment, a depth sensor (230f) can be used by the tracking component (230a) to report how far the bronchoscope 370 has entered the patient. The depth sensor (230f) can also be used to restrict possible orientations of the bronchoscope 370, and thus, the search space. It is to be understood that the depth sensor (230f) can be implemented through a computer-vision system; rather than hardware, so that hardware modifications can be kept to a minimum.

In addition, since the locations of interest are known ahead of time, final and intermediate positions of the bronchoscope 370 can be determined through the physical model (230c). This gives a list of physical instructions (230e) for the bronchoscopist to perform to reach the locations of interest, which can serve as an additional navigational aid. Anticipating a specific path and insertion steps a-prior can further constrain the possible orientations and locations for tracking. In fact, this can lead to a new goal for tracking, for example, instead of tracking the bronchoscope to provide continual updates regarding location, the goal of tracking can be to warn the bronchoscopist if he/she is off the pre-defined course.

In accordance with these embodiments, a physical model of a bronchoscope is used in combination with video-matching for both aiding a bronchoscopist in inserting a bronchoscope and further constraining possible orientations for the matching of video and virtual images. In doing so, the matching problem is greatly reduced, potentially allowing for real-time bronchoscopic tracking. In addition, this embodiment allows for the potential of greater accuracy and less manual requirements. Further, this embodiment requires little or nor change to existing equipment.

Although exemplary embodiments of the present invention have been described with reference to bronchoscopic navigation, it is to be understood that the present invention is applicable to other navigational techniques such as, but not limited to, those used for endoscopic navigation of the colon, bladder, or stomach.

It should to be understood that the present invention may be implemented in various forms of hardware, software, firmware, special purpose processors, or a combination thereof. In one embodiment, the present invention may be implemented in software as an application program tangibly embodied on a program storage device (e.g., magnetic floppy disk, RAM, CD ROM, DVD, ROM, and flash memory). The application program may be uploaded to, and executed by, a machine comprising any suitable architecture.

It is to be further understood that because some of the constituent system components and method steps depicted in the accompanying figures may be implemented in software, the actual connections between the system components (or the process steps) may differ depending on the manner in which the present invention is programmed. Given the teachings of the present invention provided herein, one of ordinary skill in the art will be able to contemplate these and similar implementations or configurations of the present invention.

It should also be understood that the above description is only representative of illustrative embodiments. For the convenience of the reader, the above description has focused on a representative sample of possible embodiments, a sample that is illustrative of the principles of the invention. The description has not attempted to exhaustively enumerate all possible variations. That alternative embodiments may not have been presented for a specific portion of the invention, or that further undescribed alternatives may be available for a portion, is not to be considered a disclaimer of those alternate embodiments. Other applications and embodiments can be implemented without departing from the spirit and scope of the present invention.

It is therefore intended, that the invention not be limited to the specifically described embodiments, because numerous permutations and combinations of the above and implementations involving non-inventive substitutions for the above can be created, but the invention is to be defined in accordance with the claims that follow. It can be appreciated that many of those undescribed embodiments are within the literal scope of the following claims, and that others are equivalent.

What is claimed is:

1. A computer-based method for bronchoscopic navigational assistance, comprising:
receiving first image data of a patient's lungs, the first image data acquired before a bronchoscopy is performed;
receiving second image data of a portion of one of the patient's lungs that includes a bronchoscope, the second image data acquired during the bronchoscopy; and
performing image registration between the first image data and the second image data to determine a global location and orientation of the bronchoscope within the patient's lung during the bronchoscopy.

2. The method of claim 1, wherein the first image data and the second image data are acquired by using a three-dimensional (3D) imaging technique.

3. The method of claim 2, wherein the first image data is a computed tomography (CT) volume.

4. The method of claim 1, wherein the second image data includes a tip of the bronchoscope.

5. The method of claim 4, wherein the second image data includes a location of a potential or actual pathology.

6. The method of claim 1, further comprising:
identifying the bronchoscope by segmenting the bronchoscope in the second image data during the bronchoscopy.

7. The method of claim 1, further comprising:
identifying an airway tree and a location of a potential or actual pathology from the first image data before the bronchoscopy is performed.

8. The method of claim 7, further comprising:
superimposing the airway tree and the location of a potential or actual pathology from the first image data onto the second image data during the bronchoscopy.

9. The method of claim 8, further comprising:
performing a virtual bronchoscopy on the second image data after superimposing the airway tree and the location of a potential or actual pathology from the first image data onto the second image data.

10. The method of claim 8, further comprising:
subtracting the bronchoscope from the second image data by segmenting the bronchoscope in the second image data during the bronchoscopy; and
performing a virtual bronchoscopy on the second image data after superimposing the location of a potential or actual pathology from the first image data onto the second image data.

11. The method of claim 10, further comprising:
fusing the first image data with the second image data.

12. A method for real-time bronchoscopic navigational assistance, comprising:
receiving image data of a patient's lungs, the image data acquired before a bronchoscopy is performed;
tracking a current global location and orientation of a bronchoscope in one of the patient's lungs by using an optical model and a physical model of the bronchoscope and real-time video of the bronchoscope during the bronchoscopy; and
automatically updating the global location and orientation of the bronchoscope in relation to the image data during the bronchoscopy.

13. The method of claim 12, wherein the image data is acquired by using a three-dimensional (3D) imaging technique.

14. The method of claim 12, further comprising:
identifying an airway tree and a location of a potential or actual pathology from the image data before the bronchoscopy is performed.

15. The method of claim 14, further comprising:
constraining a search space for a subsequent global location and orientation of the bronchoscope by using the current global location and orientation of the bronchoscope during the bronchoscopy.

16. The method of claim 14, further comprising:
constraining a search space for a subsequent global location and orientation of the bronchoscope by using a pre-selected path to the location of a potential or actual pathology during the bronchoscopy.

17. The method of claim 14, further comprising:
constraining a search space for a subsequent global location and orientation of the bronchoscope by using a segmentation of the airway tree during the bronchoscopy.

18. The method of claim 12 further comprising:
constraining a search space for a subsequent global location and orientation of the bronchoscope by using a depth sensor.

19. A system for bronchoscopic navigational assistance, comprising:
a memory device for storing a program;
a processor in communication with the memory device, the processor operative with the program to:
receive first image data of a patient's lungs, the first image data acquired before a bronchoscopy is performed;
receive second image data of a portion of one of the patient's lungs that includes a bronchoscope, the second image data acquired during the bronchoscopy; and
perform image registration between the first image data and the second image data to determine a global location and orientation of the bronchoscope within the patient's lung during the bronchoscopy.

20. The system of claim 19, wherein the first image data and the second image data are received from a three-dimensional (3D) imaging device.

21. The system of claim 19, wherein the processor is further operative with the program to:
display the global location and orientation of the bronchoscope within the patient's lung.

22. The system of claim 21, wherein the global location and orientation of the bronchoscope within the patient's lung is displayed on a computer or television monitor.

23. A system for real-time bronchoscopic navigational assistance, comprising:
a memory device for storing a program;
a processor in communication with the memory device, the processor operative with the program to:
receive image data of a patient's lungs, the image data acquired before a bronchoscopy is performed:
track a current global location and orientation of a bronchoscope in one of the patient's lungs by using an optical model and a physical model of the bronchoscope and real-time video of the bronchoscope during the bronchoscopy; and
automatically update the global location and orientation of the bronchoscope in relation to the image data during the bronchoscopy.

24. The system of claim 23, wherein the image data is received from a three-dimensional (3D) imaging device.

25. The system of claim 23, wherein the processor is further operative with the program to:
display the automatically updated global location and orientation of the bronchoscope within the patient's lung.

26. The system of claim 25, wherein the global location and orientation of the bronchoscope within the patient's lung is displayed on a computer or television monitor.

27. A computer-based method for endoscopic navigational assistance, comprising:
receiving first image data of region of interest inside a patient, the first image data acquired before an endoscopy is performed;
receiving second image data of a portion of the region of interest that includes an endoscope, the second image data acquired during the endoscopy; and
performing image registration between the first image data and the second image data to determine a global location and orientation of the endoscope within the region of interest during the endoscopy.

28. A method for real-time endoscopic navigational assistance, comprising:
receiving image data of a region of interest inside a patient, the image data acquired before an endoscopy is performed:
tracking a current global location and orientation of an endoscope in a portion of the region of interest by using an optical model and a physical model of the endoscope and real-time video of the endoscope during the endoscopy; and
automatically updating the global location and orientation of the endoscope in relation to the image data during the endoscopy.
